(19) United States
(12) Patent Application Publication
(10) Pub. No.: US 2014/0329715 A1
(43) Pub. Date: Nov. 6, 2014

(54) Water Treatment and Monitoring

(71) Applicant: Heriot-Watt University, Edinburgh (GB)

(72) Inventors: Helen Bridle, Edinburgh (GB); Mark Bradley, Edinburgh (GB); Mei Wu, Edinburgh (GB)

(21) Appl. No.: 14/361,663

(22) PCT Filed: Nov. 29, 2012
(86) PCT No.: PCT/GB2012/052939

§ 371 Date: May 29, 2014

(30) Foreign Application Priority Data
Nov. 29, 2011 (GB) 1120496.3

(51) Int. Cl. B01J 20/26; G01N 33/569; C02F 1/28

(52) U.S. Cl. CPC B01J 20/261; C02F 1/285; G01N 33/56905; B01J 20/264; B01J 20/262
USPC 506/9; 210/660; 526/307.8; 526/312; 524/555; 528/77; 524/590; 528/85; 528/70

(57) ABSTRACT

The present invention relates to the provision of polymers that are selected to have either; surface properties that allow protozoa, in particular Cryptosporidium and Giardia, to bind to the polymer; or have surface properties that are repellent to the binding of these protozoa. Methods for identifying suitable polymers are provided. Products comprising, consisting of or coated with the polymers of the present invention are also provided, as well as methods of treating or monitoring water employing polymers of the present invention.

### FIELD OF THE INVENTION

The present invention relates to the provision of polymers that are selected to have either; surface properties that allow protozoa, in particular Cryptosporidium and Giardia, to bind to the polymer; or have surface properties that are repellent to the binding of these protozoa. Methods for identifying suitable polymers are provided. Products comprising, consisting of, or coated with the polymers of the present invention are also provided, as well as methods of treating or monitoring water employing polymers of the present invention.

### BACKGROUND TO THE INVENTION

Contamination of water by protozoa, such as Cryptosporidium and Giardia is a serious global issue. These pathogens are ubiquitous in the environment, resistant to standard chlorination disinfection procedures and have a low infectious dose (1). For this reason, regulatory monitoring is frequently undertaken. However, the existing protocols have low recovery rates (13% is considered acceptable) and does not provide information on species of viability of the detected pathogens.

Ingestion of human pathogenic species of Cryptosporidium oocysts causes cryptosporidiosis, for which there is no safe and effective treatment, and ingestion of Giardia cysts causes giardiosis. In developing countries, it is estimated that 250-500 million cryptosporidiosis cases occur each year, playing a significant role in high childhood mortality and morbidity. Prevalence of giardiosis is around 20-30% in the developing world, with up to 100% of children acquiring the infection before the age of 3. In the developed world, where water treatment is better and more wide-spread, the prevalence is lower but outbreaks do occur. For Cryptosporidium one of the most serious outbreaks was in Milawaukee in 1993, and there were several recent cases in the UK, Australia and in Sweden. In the US Giardia was the most common intestinal protozoan infection in the early 2000s with infections reported in Norway in 2004.

Understanding the behaviour and fate of protozoa in water treatment systems is essential to assess risk at existing plants and appropriately design future systems. Although it is known that the nature of the coagulation pretreatment is very important for the efficiency of the subsequent water treatment processes, the exact adhesion and removal mechanisms have not been elucidated. Few field studies of protozoa in water treatment systems have been undertaken, due to limitations in assay techniques for determining a mass balance for (oo)cysts and lack of understanding of the mechanisms of interaction with chemicals or surfaces within the process. Instead, laboratory studies have concentrated on the adhesion characteristics, to a range of materials, and measurement of interaction forces.

While various studies of Cryptosporidium adhesion have been undertaken, with materials ranging from metal oxides, quartz, silanes, natural organic matter, biofilms, clays and natural suspended sediments, little work, apart from a paper by Dai et al have investigated polymeric materials (2). The majority of studies investigating Giardia interactions with surfaces have focused on the post-ingestion trophozoite stage and its attachment through an adhesive disk. There has been limited investigation of the cyst stage, where the adhesive disk is internalised and fragmented, apart from the Dai paper.

Although the Dai paper looks at the adhesion of both Cryptosporidium and Giardia to various surfaces, its teaching suggests that due to the difference in the surface properties of the two different organisms, polymers are likely to respond differently, in terms of binding Cryptosporidium and Giardia and hence it may not be expected to identify a polymer which could be used to bind or repel both types of organism. Also, the Dai paper appears to suggest that polystyrene may bind, rather than repel Cryptosporidium and Giardia, but to differing degrees.

It is amongst the objects of the present invention to obviate and/or mitigate one or more of the aforementioned disadvantages.

It is a further object of the present invention to provide one or more polymers which may be of use in attracting or repelling one or more species of protozoa.

### SUMMARY OF THE INVENTION

The present invention is based on the identification (by use of polymer microarrays) of polymers, especially polyacrylate/polyacylamide and polyurethane polymers, that exhibit distinctive binding or repellent (non-binding) properties towards protozoa, especially Cryptosporidium or Giardia. The polymers can be selective in their binding properties, for example binding viable protozoa in preference to or even whilst being repellent to non-viable protozoa.

Thus in a first aspect, the present invention provides a method for identifying polymers which are capable of binding to, or are poorly binding and hence may be considered as non-binding to protozoa, such as Cryptosporidium or Giardia comprising:
providing a library of polymer samples;
exposing the polymer samples to a target protozoa, such as a Crytposporidium or Giardia; and
observing binding or non-binding of the target protozoa species to the polymer samples.

Advantageously, the polymer samples are made by high throughput methods such as parallel synthesis techniques or inkjet printing. Advantageously, the testing is carried out by preparing micro-arrays of polymer samples which are then exposed to the protozoa. Desirably the method may allow the identification of a polymer which is able to bind or repel two or more different species and/or genus of protozoa. For example, the method may allow identification of a polymer which is able to bind or repel Cryptosporidium and Giardia species.

In a further aspect, the present invention provides one or more polymers which are able to bind or repel target protozoa, such as Cryptosporidium and/or Giardia. Such polymers can have a number of uses.

The polymers provided in the following description may be used in a number of applications where the ability to bind to protozoa or to repel or at least only weakly bind to protozoa is useful. The protozoa may be viable or non-viable. For example the polymers may bind or repel non-viable protozoan (oo)cysts as well as viable protozoa. Particularly preferred applications relate to the treatment or monitoring of water, so that protozoa, such as Cryptosporidium/Giardia, may be removed or isolated from samples of water. In certain applications it may be desirable to bind protozoa and in other applications it may be desirable to prevent or minimise binding of protozoa. Indeed a whole process may include one or more components designed to bind protozoa and one or more components designed to repel or reduce or prevent binding of protozoa.

The present invention provides uses (i.e. methods of using) of the polymers described herein including methods of binding protozoa to a substrate e.g. the surface of a substrate and methods of preventing binding of protozoa to a substrate e.g. the surface of a substrate. Selective binding of particular organisms may also be achieved. The methods can be applicable in a wide range of technologies including protozoal detection or filter systems, water delivery and treatment; medical devices and appliances; food industry and related technologies.

Thus the present invention provides an article comprising, consisting of, consisting essentially of, or coated with a protozoa binding polymer or a protozoa non-binding polymer as described herein.

Thus the present invention in one embodiment provides a coating for a substrate, the coating comprising, consisting of or consisting essentially of a protozoan binding polymer or a protozoan non-binding polymer as described herein.

The present invention also provides protozoa binding polymers or protozoa non-binding polymers as described herein. The polymers may be used in manufacture of an article or a coating for a substrate. Such polymers may also include antimicrobial agents, chemical agents and/or enzymatic substrates within the polymers.

In general the non-binding polymers may be used to avoid binding or fouling by protozoa. For further example the polymers may be used in methods of preventing or avoiding fouling of water systems such as water supply systems, heating and cooling units, swimming pools and their water treatment systems and storage tanks. Fouling by protozoa will be resisted by making use of the non-binding polymers as coatings or constituents of pipes, pumps storage tanks and the like, plaster, paints, and other construction fabrication materials or other surface coatings. Paints and other coatings applications may include protective/anti-biofouling coatings for use in swimming pools, water tanks and systems.

Yet further uses of the non-binding (repellent) or selectively binding polymers can be envisaged for consumer products (from personal hygiene and household cleaning to food stuffs). The use of non-binding (repellent) polymers in household cleaning products and repellent food packaging, may be envisaged.

Binding polymers may be used, for example, in, or as part of an article, which may be used as a filter for water or biological fluids. The filter may include filter media comprising, consisting, consisting essentially of, or coated with a binding polymer of the invention. On passage of a fluid contaminated with an undesired protozoan e.g. Cryptosporidium, the contaminating protozoan becomes attached to the binding polymer.

In one embodiment, the polymers of the present invention may be coated on to the surface of beads, such as polymer or glass beads known in the art. Such beads may be used to capture (e.g. undesirable) Cryptosporidium/Giardia and in this regard the water may be treated, or this may facilitate monitoring of the water, as any protozoa bound to the beads may easily be isolated and/or detected.

In certain embodiments, the binding may occur under one defined condition (e.g. at a particular pH) and the protozoa may be released by changing the condition from the condition used for binding (e.g. altering the pH).

In another embodiment, for example, the article may be a swab including a binding polymer and used to swab areas that may be contaminated. The swab can be tested, directly or indirectly for the presence of protozoa or their components. The swab can assist in disinfecting the area by virtue of the binding of contaminating protozoa. Thus the swab can disinfect an area, for example a work surface.

By way of further example the article may be a cleaning material such as a cloth or a liquid (that may be a suspension or a solution) that contains binding polymers for rapid scavenging of protozoa. Selective binding polymers can be used to collect or scavenge harmful protozoa (e.g. viable, species specific) whilst leaving non harmful or beneficial protozoa unaffected or relatively unaffected.

By way of further example, the article may be a contained or slow release structure for use in agriculture, or in human or animal subjects. Such articles may be used for bio-control, use in vaccination or therapy, or for bioconversion or bio-production. Protozoa (e.g. non-viable) adhered to a binding polymer surface in such an article may themselves, or components from them, be released gradually—thus providing controlled release in a human or animal subject, for example.

In one embodiment of the invention polyacrylate/polyacrylamide polymers that show strong binding to protozoa are formed in a polymerisation reaction. Typically the polymers may be formed from polymerisation of one or more (typically two or three) monomers, which may be present in differing amounts. Preferably the, or one of the monomers is a substituted alkyl methacrylate or acrylate monomer. The alkyl group of the substituted alkyl function may have from 1 to 10 or even from 1 to 4 carbon atoms and the substituent group may be a alkoxy group with 1 to 4 carbon atoms, such as a methoxy group, or a dialkylamino group, such as dimethylamino. Particularly preferred monomers are MEMA (2-methoxymethacrylate), DEAEMA (2-(diethylamino)ethyl methacrylate) and MEA (2-methoxyacrylate). Preferred polymers comprise two or more monomers and typically a mixture of MEMA or MEA, and DEAEMA, in a ratio of 30:70 to 70:30, such as 50:50 to 80:20 respectively. Particularly preferred ratios of MEMA or MEA to DEAEMA are 70:30 and more preferably 55:45 or 50:50 respectively. Optionally more monomers may be present, such as a further monomer, such as acrylic acid (A-H). A suitable polymer includes MEMA, DEAEMA and A-H in a ratio of 50:45:5 to 70:25:5, such as 60:30:10. All ratio mentioned herein are in mol % terms.

Three polymers having the following monomer combinations: MEMA(55%):DEAEMA(45%); MEMA(70%):DEAEMA(30%); and MEMA(60%):DEAEMA(30%):A-H(10%) have been shown to bind well to Cryptosporidium, such as Cryptosporidium parvum. 3 polymers having the following monomer combinations: MEMA(55%):DEAEMA(45%); MEA(50%):DEAEMA(50%); and MEMA(60%):DEAEMA(30%):A-H(10%) have been shown to bind well to Giardia, such as Giardia lamblia. Advantageously, the present invention provides polymers which are able to bind strongly to both Cryptosporidium and Giardia species. Typically such polymers may comprise MEMA and DEAEMA in an amount of 50-60:50-30%, with a further 10% (where necessary), comprising A-H. Results of binding assays with Cryptosporidium are discussed hereafter. As is known by the skilled person terms such as good binding and poor binding are relative and will depend on the conditions employed, the protozoan strain and the manner and place in which the polymer is applied. The testing regime applied is described hereafter in the Detailed Description of the Invention.

In another embodiment the present invention provides acrylate or acrylamide/vinyl polymers, or polyurethane polymers that are repellent (weakly/poorly bind) to protozoa, such as Cryptosporidium and/or Giardia. Preferably the polymers are repellent to both Cryptosporidium and Giardia. Such polymers may comprise one or more monomers which include an aryl group, such as styrene (St) and optionally a dialkylacrylamide group (alkyl representing 1 to 4 carbon atoms), such as dimethylacrylamide (DMAA); and diethylacrylamide (DEAA). Preferably the polymers comprise two monomers selected from styrene and a dialkylacrylamide. Preferred polymers comprise, or consist of the following monomers: St:DMAA and St:DEAA and which may be present in the following ratios 45:55 to 95:5, such as 50:50 to 90:10, for example 50:50, 70:30 or 90:10.

Additional polymers which may weakly bind Giardia species may comprise MEA (2-methoxyacrylate) and a dialkylacrylamide group (alkyl representing 1 to 4 carbon atoms), such as dimethylacrylamide (DMAA); and diethylacrylamide (DEAA). Preferred polymers comprise or consist of MEA:DMAA and MEA:DEAA, which may be present in the following ratios 45-75:55-25% respectively, such as 50-70:50-30%. Particularly preferred polymers are MEA(50%):DEAA(50%) and MEA(70%):DMAA(30%).

In another embodiment the invention provides polyurethane polymers that are non-binding/repellent to protozoa and are polyurethane polymers formed by polymerising a polydiol with a di-isocyanate and optionally with an extender molecule, such as a diol. The extender molecules have the effect of modifying the physical character of the polymers, for example, polymer shape, viscosity and polymer state. Such polyurethane polymers have been found to bind poorly e.g. repel or prevent binding of both Cryptosporidium and Giardia species.

The polydiol may be selected from the group consisting of: PPG-PEG: poly(propylene glycol)-poly(ethylene glycol); PTMG: poly(tetramethylene glycol) also known as poly(butylene glycol); and PHNAD: poly[1,6-hexanediol/neopentyl glycol-alt-(adipic acid)]diol.

The molecular weights of the polydiol may be from Mn=600 to Mn=2500 and may be present in an amount of 15-55%, such as 20-50% of the polymer.

The di-isocyanate may be selected from the group consisting of:

HDI: 1,6-diisocyanohexane;

MDI: 4,4′-methylenebis(phenylisocyanate); and

BICH: 1,3-bis(isocynanatomethyl)cyclohexane.

Typically the di-isocyanate is present in an amount of 45-55% of the polymer.

Suitable extenders include:

BD: 1,4-butanediol;

OFHD: 2,2,3,3,4,4,5,5-octafluoro-1,6-hexanediol; and

DMAPD: 3-dimethylamino-1,2-propanediol.

When present, the extender may be present in an amount of 10-30 mol % of the polymer, typically 15-25%.

Preferred compositions of polyurethane polymers exhibiting binding to bacteria are listed in Table A below. Results of binding assays are discussed hereafter:

TABLE A
Polyurethanes which display poor binding (non-binding/repellent) to
Cryptosporidium and Giardia

| PU | Polymer Structure | | | | | | |
|---|---|---|---|---|---|---|---|
| reference | | | | | Ratio (mol) | | |
| number | Diol | Mn | Dis. | Ext. | Diol | Dis | Ext |
| PU91 | PTMG | 650 | MDI | none | 48.5 | 51.5 | 0 |
| PU223 | PHNAD | 1800 | BICH | OFHD | 25 | 52 | 23 |
| PU226 | PHNAD | 1800 | MDI | none | 48.5 | 51.5 | 0 |
| PU230 | PPG-PEG | 1900 | HDI | BD | 25 | 52 | 23 |
| PU239 | PPG-PEG | 1900 | HDI | DMAPD | 25 | 52 | 23 |

[Diol = polydiol as listed above; Mn = molecular weight (number average) of polydiol; Dis = diisocyanate; Ext. = extender].

Particularly preferred embodiments of the present invention are concerned with water purification at both a personal and industrial scale. On the person scale, devices may be provided which may include, for example, filters which include one or more surfaces coated with a binding polymer of the present invention, such that protozoa e.g. Cryptosporidium binds to the coated surface and may be removed in order to allow purification of the water. At the industrial scale, water treatment plants may include parts of a system which may be formed from or coated with non-binding polymers of the present invention and other parts which may be coated/formed from binding polymers of the present invention. For example, it may be desirable that certain pipes or tubing does not bind protozoa and hence is kept essentially free of protozoa and hence non-binding polymers of the present invention would be employed. However, other parts of a water treatment system which act to filter out undesirable material could include the binding polymers of the present invention, so as to bind and hence remove protozoa from the water being purified. Typically, filters are used to remove microorganisms such as protozoa, including Cryptosporidium from water and filters may be provided which include portions thereof formed from, comprising, or coated with binding polymers of the present invention. Membrane filters may be provided in this manner, or the binding polymers may, for example, be made of, or coated on the surface of particles or beads and may be used as a component of “sand” filters, known in the art.

For monitoring of, for example, water quality, filters make be used which are formed from, comprise and/or are coated with the non-binding polymers described herein. In this manner, the filter may be used to physically trap protozoa, such as Cryptosporidium and/or Giardia, but by virtue of the non-binding nature of the polymers may be readily released from the filter after trapping and hence the presence or otherwise of the protozoa in the water, can readily be tested or monitored.

### DETAILED DESCRIPTION

The present invention will now be further described by way of example and with reference to the figures which show:

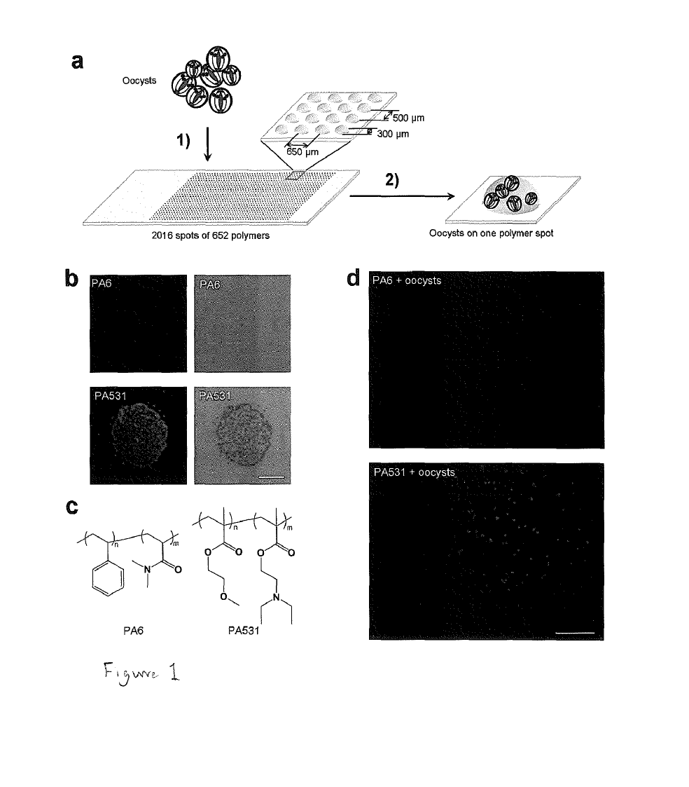

FIG. 1. Array screening for Cryptosporidium (in this case with the species C. parvum) oocyst binding. (a). Oocysts (1 million) were incubated for 3 hrs on the polymer microarray. Adhesion to the polymers was analysed by high-content imaging (n=3). (b) Images of the polymer features binding viable C. parvum with oocysts stained with Crypto-a-glo (green fluorescence), and DAPI (blue fluorescence). Fluorescent (left) and phase contrast (right) images of one polymer feature selected from a poor binding polymer (PA6) and a strongly binding polymer (PA 531). (c) Chemical structures for the two polymers. d) Viable oocysts on the polymer surface of PA6 and PA531 coated coverslips. Scale bars are 100 μm in (b) and (d).

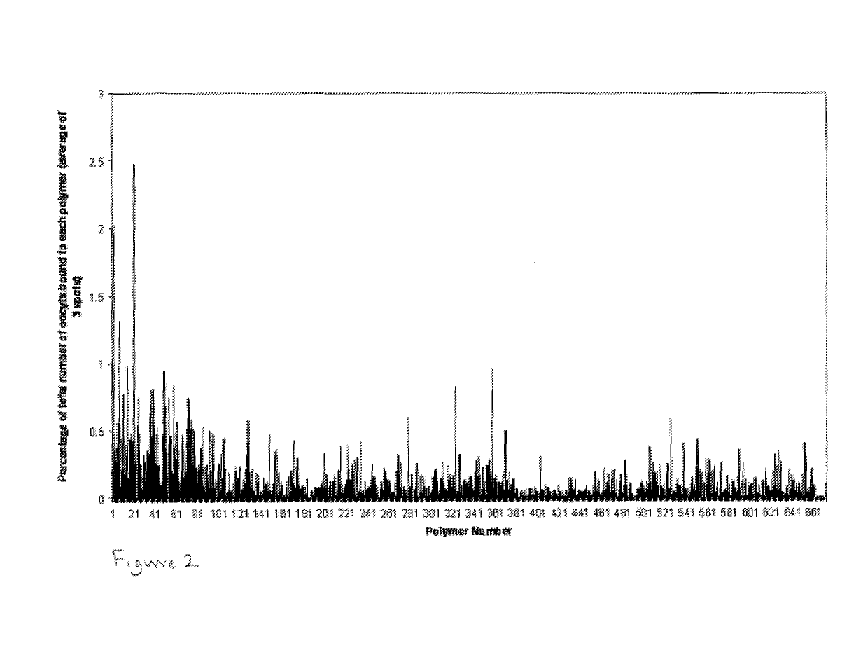

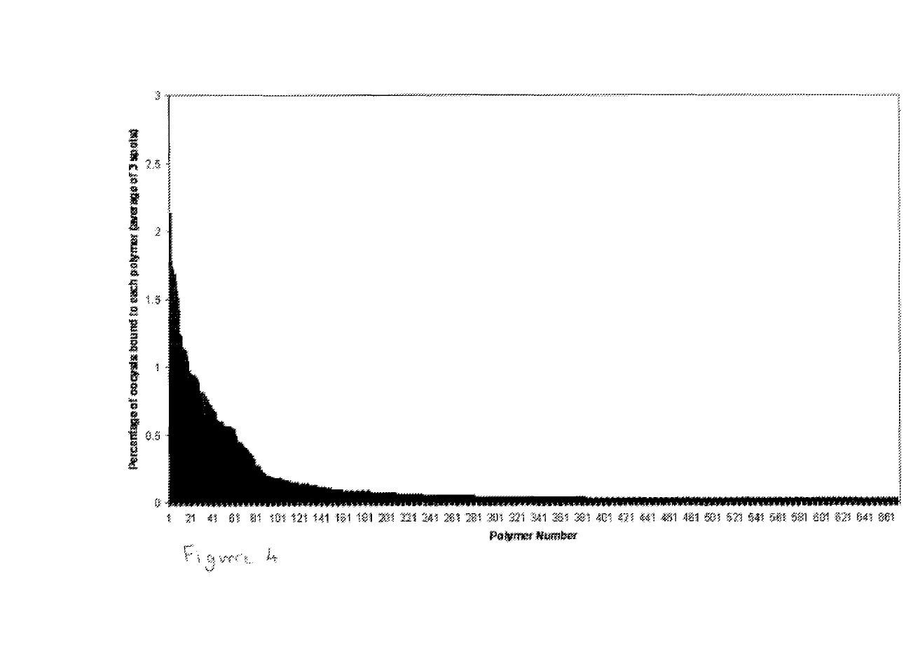

FIG. 2. Results of the viable C. parvum oocysts initial polymer microarray screen (normalised as a percentage of the total oocyst count). The graph clearly illustrates large variation in the polymer adhesion characteristics across different materials. The materials are numbered 1 to 672 (652 polymers and 20 controls) in the same order in FIGS. 2 and 4. From comparison of 2 and 4, oocyst viability clearly influences adhesion.

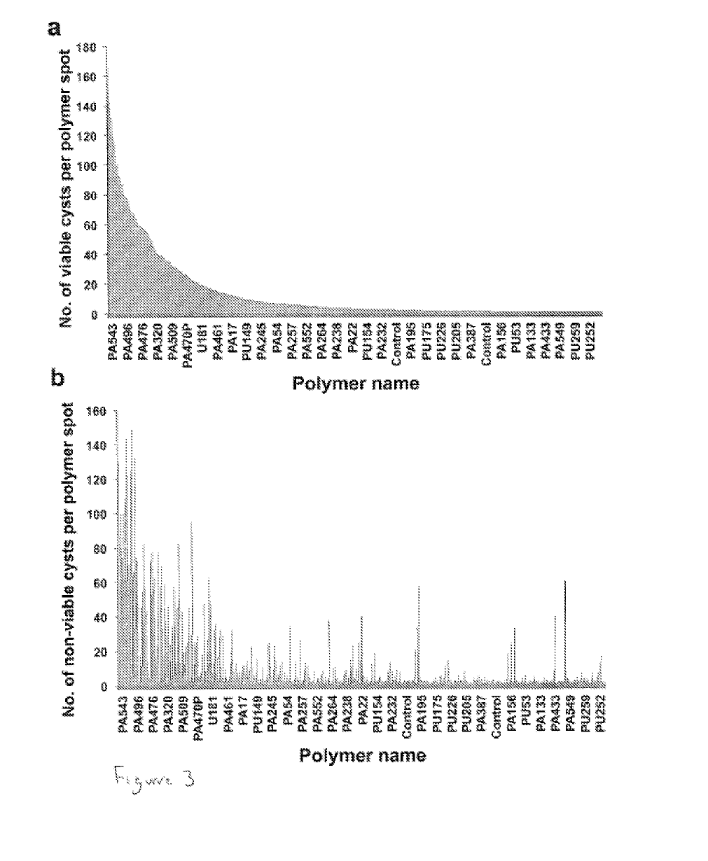

FIG. 3. Initial array screening of G. lamblia cyst binding. (a). Graph of viable cyst binding with polymers ranked in order of strongest binding, from left to right. (b). Graph of non-viable cyst binding with polymers ranked using the order from (a) to compare with viable cysts.

FIG. 4. Results of the non-viable C. parvum oocysts initial polymer microarray screen (normalised as a percentage of the total oocyst count and ordered from best to worst binding performance). The graph clearly illustrates large variation in the polymer adhesion characteristics across different materials. The materials are numbered 1 to 672 (652 polymers and 20 controls) in the same order in FIGS. 2 and 4. From comparison of 2 and 4, oocyst viability clearly influences adhesion.

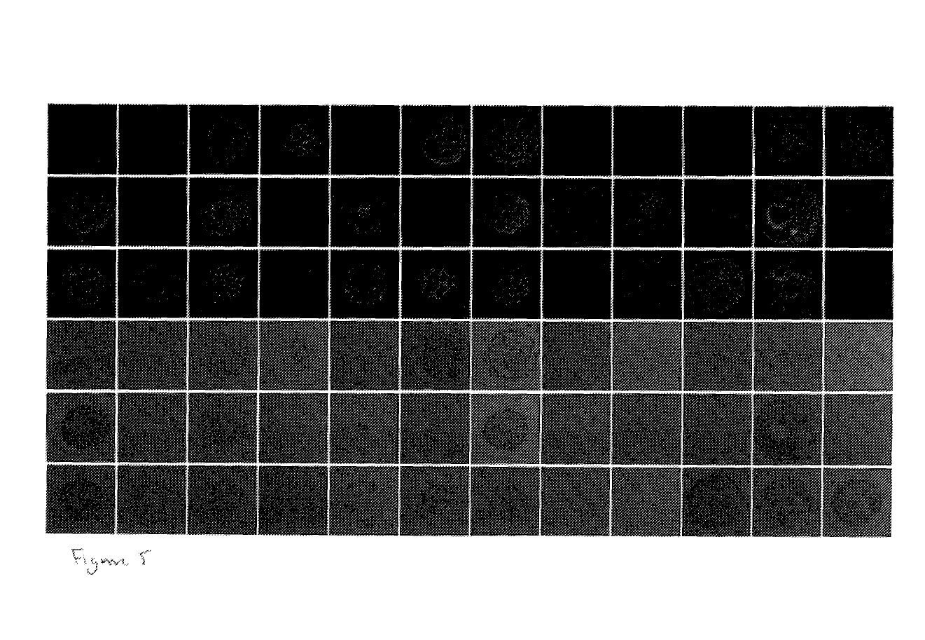

FIG. 5. Image of the hit polymer array with 34 polymers tested with viable C. parvum, (left) fluorescence and (right) phase contrast. Only one spot per polymer is shown.

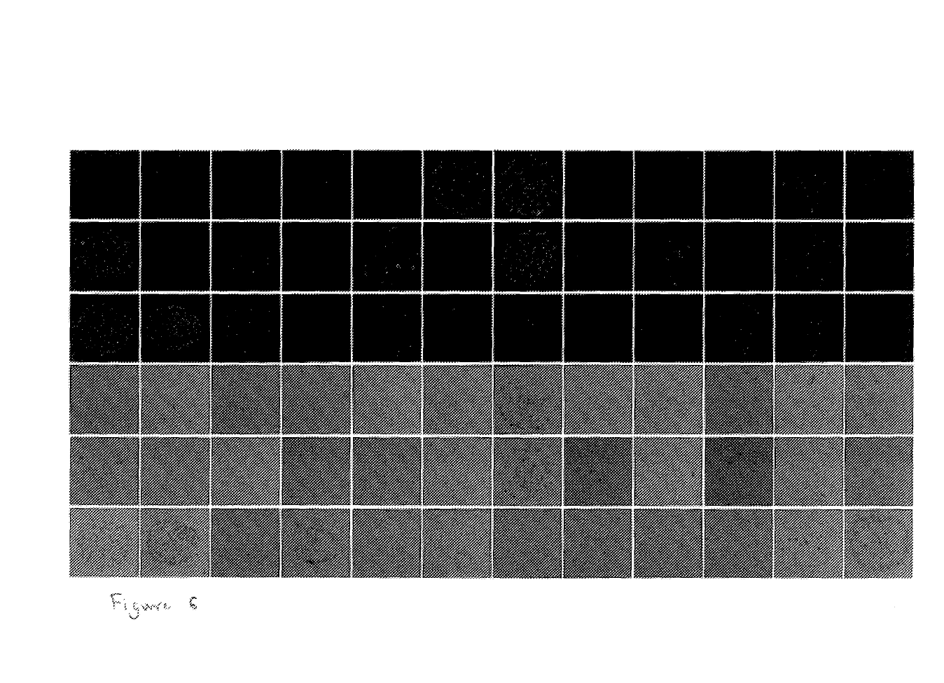

FIG. 6. Image of the hit polymer array with 34 polymers tested with non-viable C. parvum, (left) fluorescence and (right) phase contrast. Only one spot per polymer is shown.

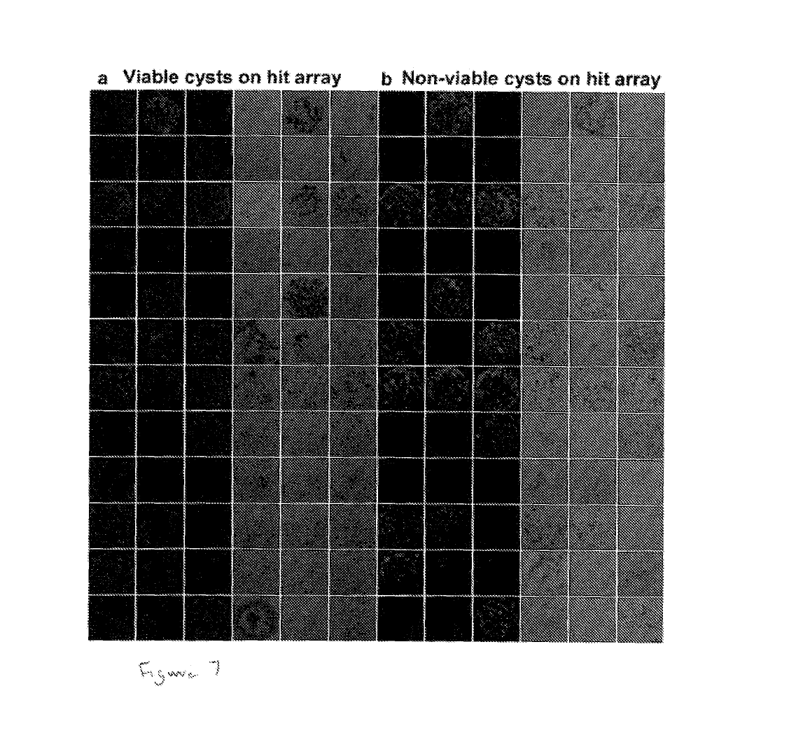

FIG. 7. Complete G. lamblia hit arrays. Images of the cysts stained with Giardia-a-glo (green fluorescence), and DAPI (blue fluorescence) bound to polymer spots. (a) Fluorescent (left) and phase contrast (right) images of selected polymers are shown for the viable hit array. (b). Fluorescent (left) and phase contrast (right) images of selected polymers are shown for the non-viable hit array.

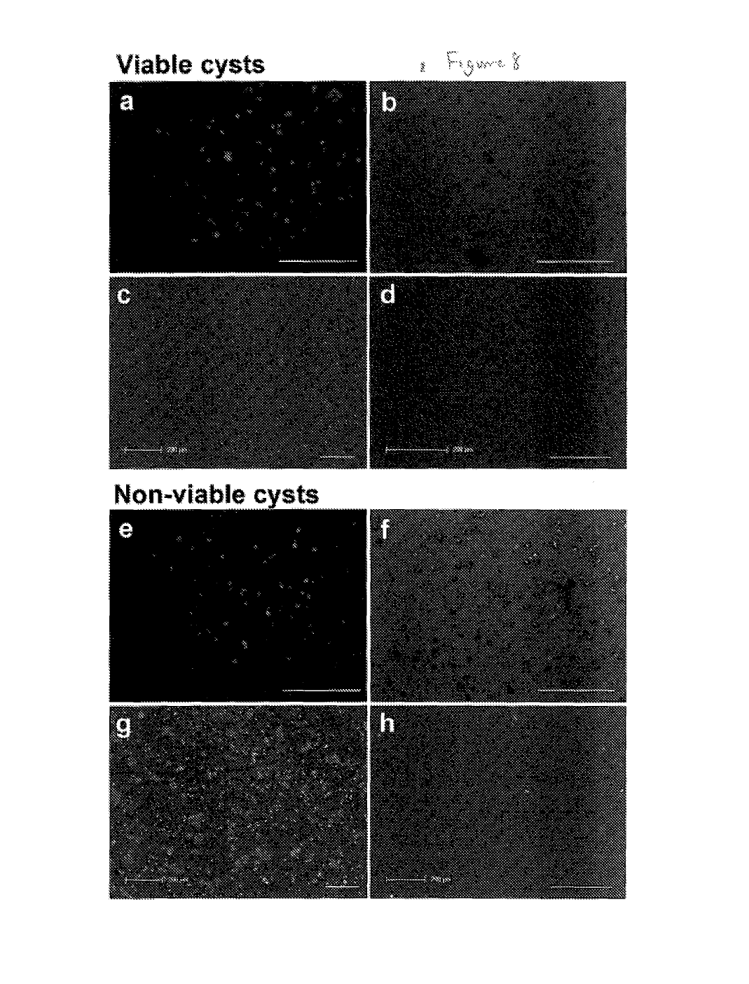

FIG. 8. Polymer scale-up screening for G. lamblia cyst binding.

(a-c). Fluorescence, phase contrast and SEM images of viable cysts on PA104.

(d). SEM image of viable cyst binding on PA6. (e-g). Fluorescence, phase contrast and SEM images of non-viable cysts on PA104. (h). SEM image of non-viable cyst binding on PA6. For the fluorescence images cysts were stained with Giardia-a-glo (green), and DAPI (blue). Scale bar=200 μm.

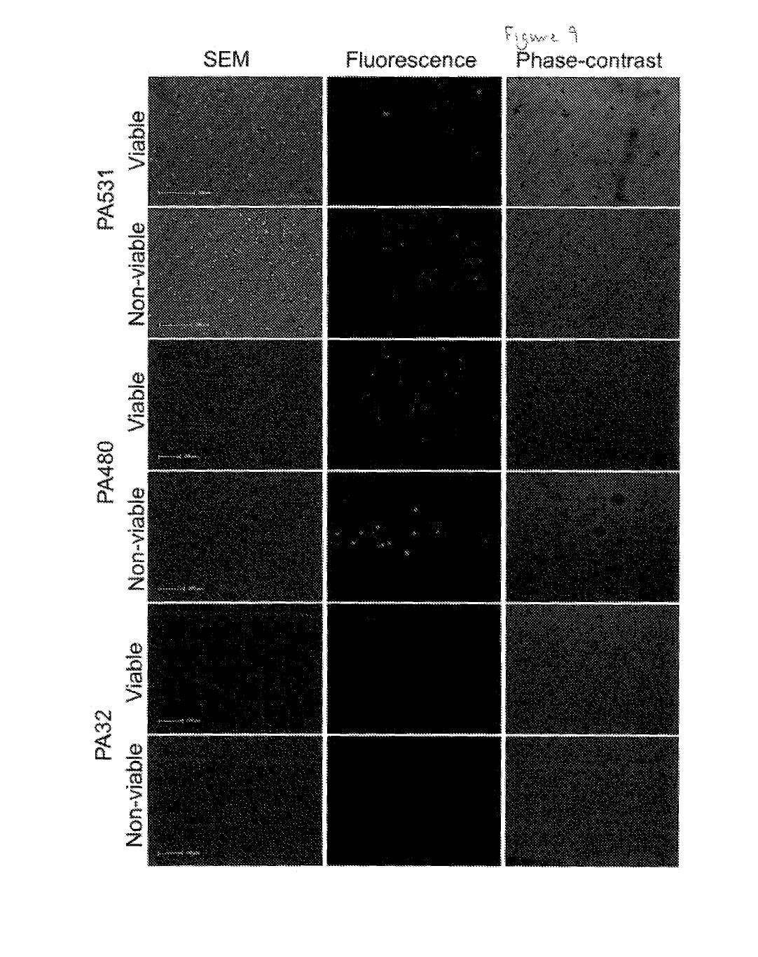

FIG. 9. SEM, fluorescence and phase contrast images of the scale-up results of various polyacrylate polymers at for both viable and non-viable G. lamblia cysts.

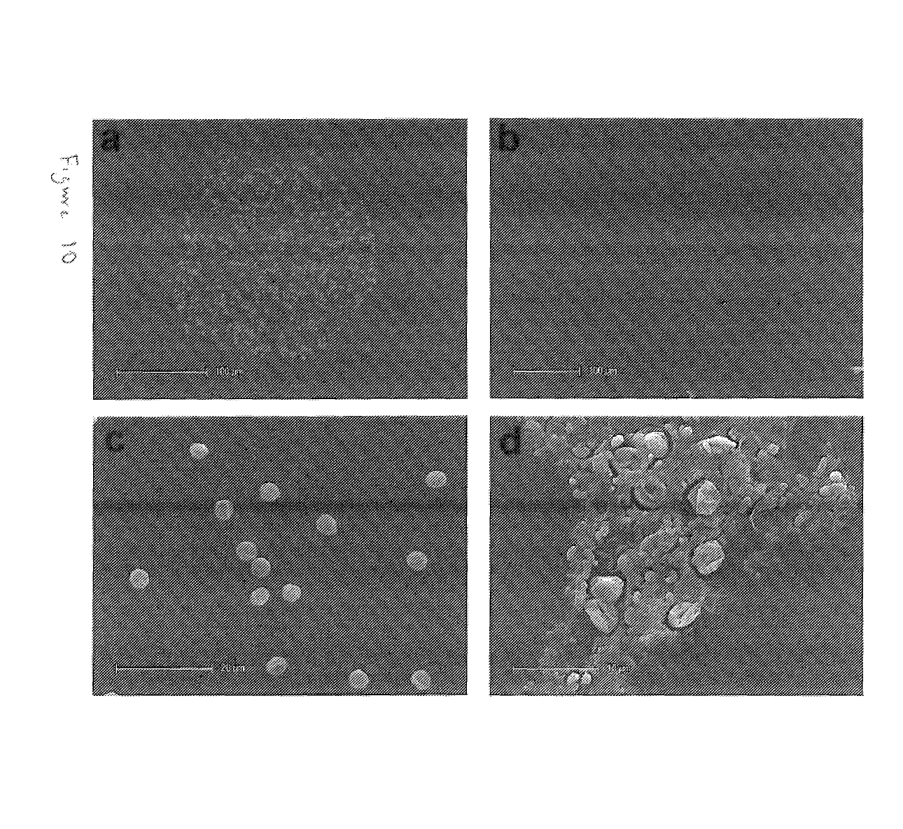

FIG. 10. SEM images of viable/non-viable C. parvum oocysts binding on selected polymers. (a) Viable cell attachment on the strong binding polymer PA531; (b) Negligible viable cell attachment on the poor binding polymer PA6; (c) Morphology of viable oocyst attachment on PA531 coated glass surface. (d) A proportion of non-viable cells adhering to the surface, showing excystation expelling their internal sporozoites. Scale bars are shown in (a) to (d).

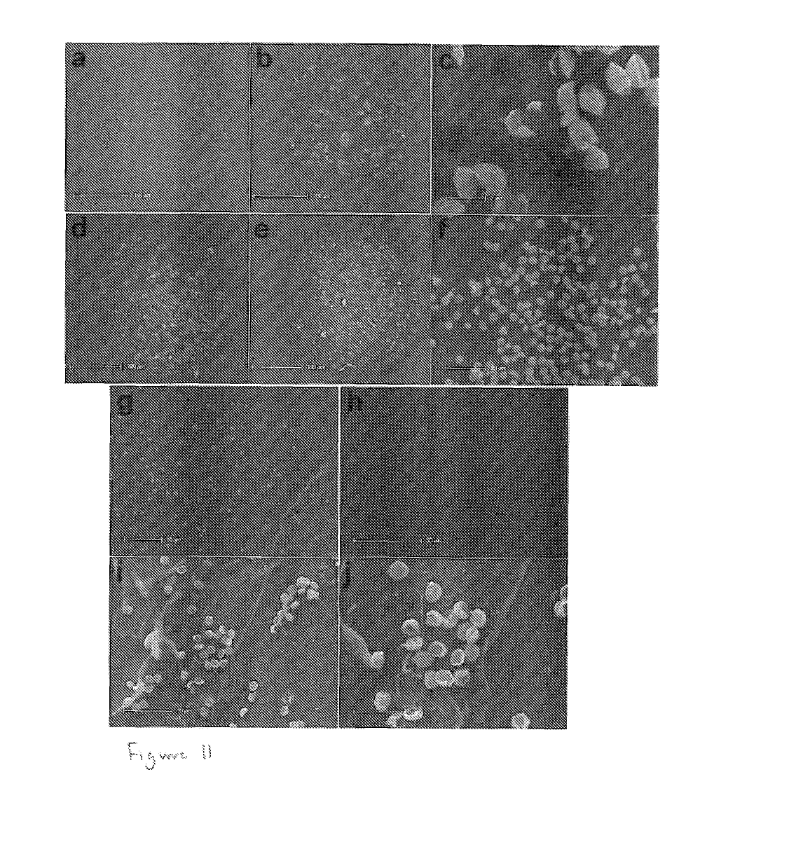

FIG. 11. SEM images of viable/non-viable C. parvum oocysts on selected polymers on the hit array (a-f) and coated substrates (g-j). (a) Viable cell attachment on the strong binding polymer spot, PA113; (b) Non-viable cell adherence on PA113; (c) Morphology of the non-viable oocyst attachment on PA113; (d) Significant viable cell attachment on the strong binding polymer spot, PA480; (e) Non-viable oocysts adhered on PA480 spot; (f) Morphology of viable oocysts binding on PA480. (g) Viable oocyst attachment on the strong binding polymer PA531 coated surface (different area to shown in FIG. 4). (h) Viable oocysts did not attach on the poor binding polymer PA6 coated surface. (i) Non-viable oocysts adhered on PA504 coated surface. (j) Morphology of non-viable oocysts binding on PA504 coated surface. Scale bars are shown to (a) to (j).

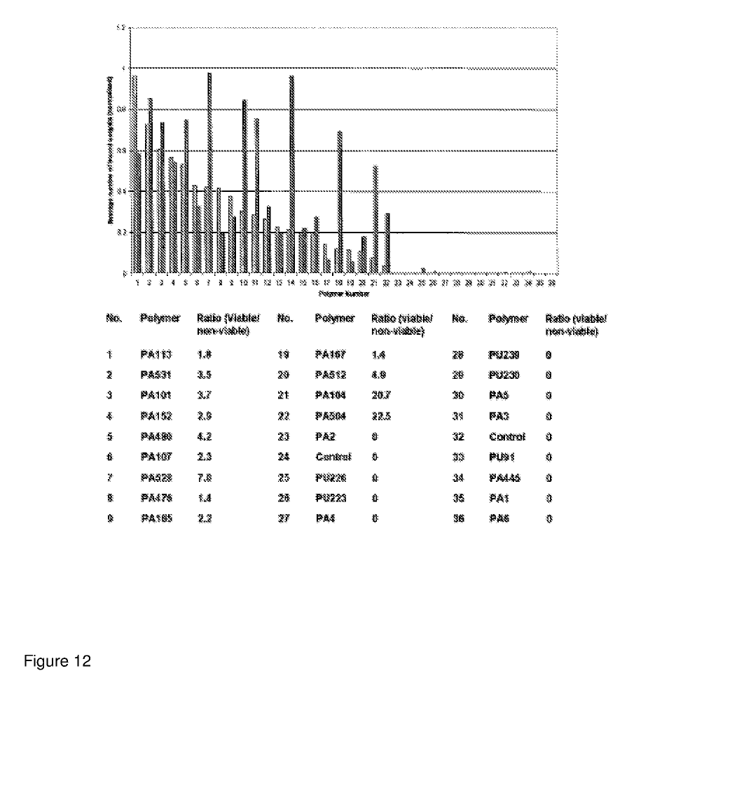

FIG. 12. A) Bar chart indicating the average number of bound C. parvum oocysts for each polymer (averaged over the 5 spots). Binding is expressed as background corrected mean fluorescent intensity. Blue: non-viable oocysts (normalized by dividing the number of oocysts by 100). Red: viable oocysts (normalized by dividing the number of oocysts by 300). X-axis: polymer code. Y-axis: fluorescent intensity in arbitrary units (au). B) Table indicating which polymers are referred to by the numbers 1-36 in the above bar chart. The ratio column compares the number of bound viable oocysts to the number of bound non-viable oocysts.

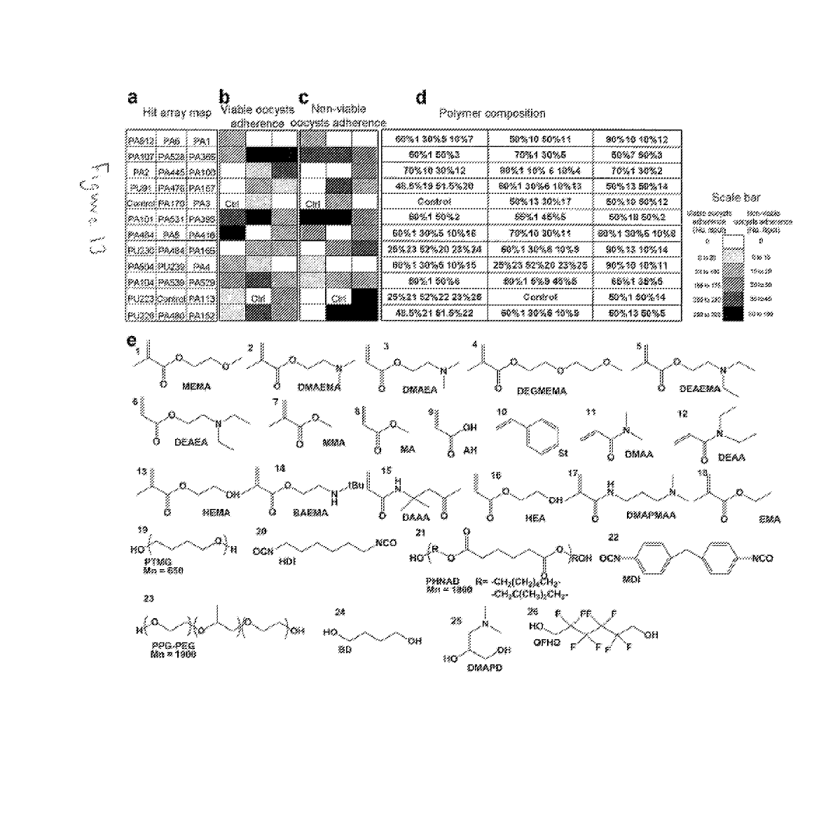

FIG. 13. Mapping the binding behaviour of viable and non-viable C. parvum oocysts. (a) Location map of the 34 selected polymers. (b)/(c) Viable/non-viable oocysts adherence on the arrays. (d) Composition of the polymers, with the monomer structures shown in (e).

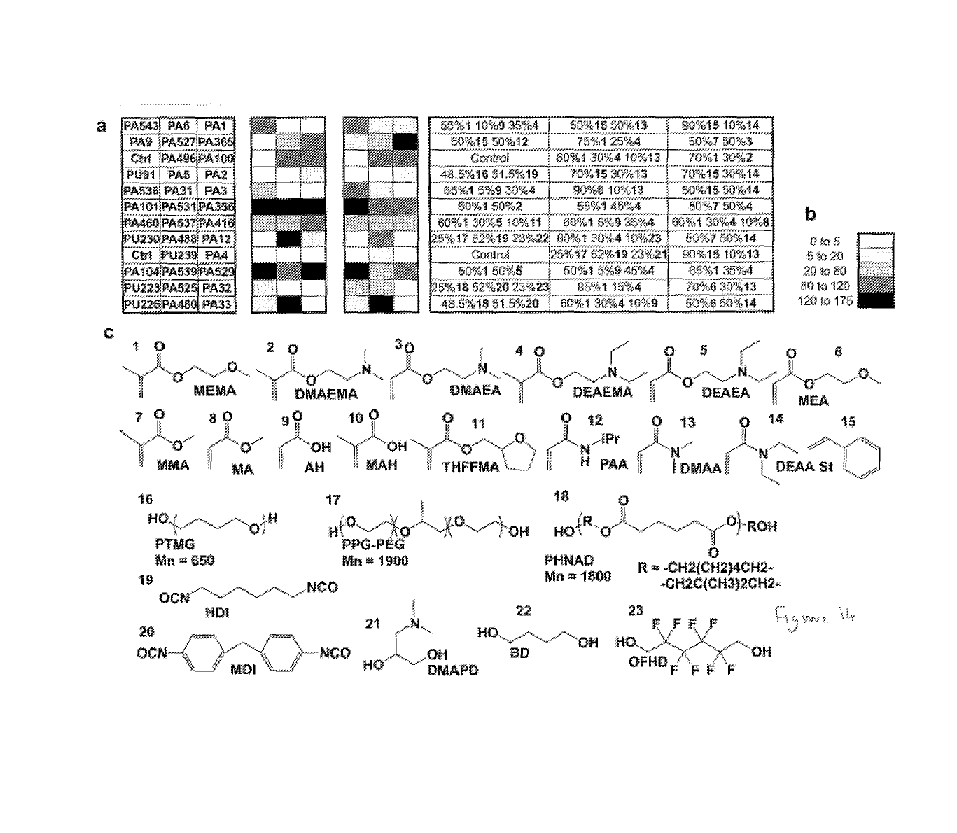

FIG. 14. Analysis of G. lamblia microarray results and polymer structures.

(a). Left to right: the polymers identity; the binding of viable; and non-viable cysts respectively; and the polymer composition.

(b). Bars relating colour intensity to cyst binding.

(c). Chemical structures of the monomers.

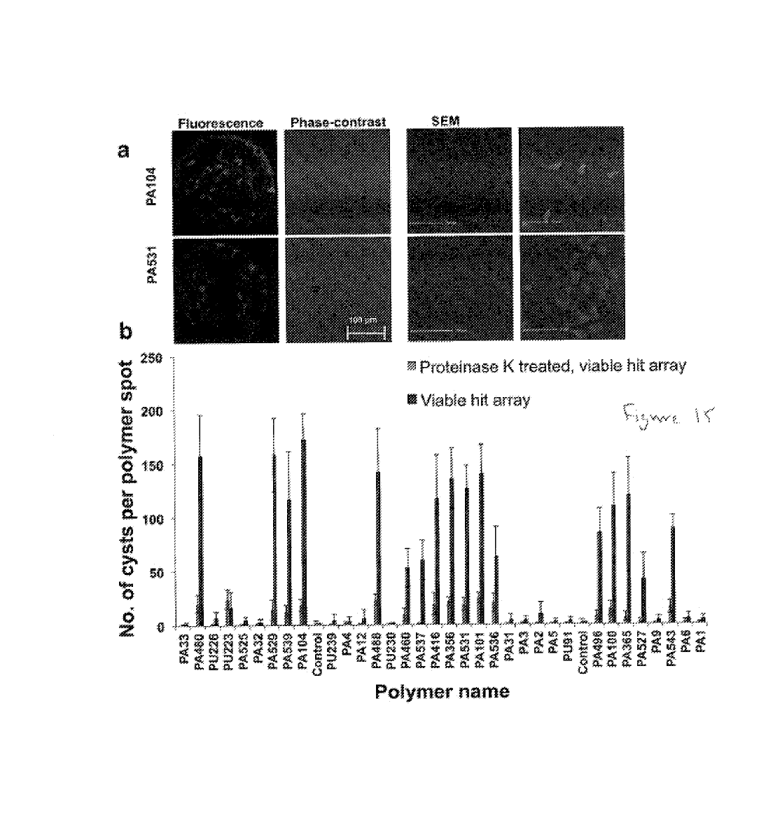

FIG. 15. Proteinase K treated G. lamblia hit array.

(a). Images of the cysts stained with Giardia-a-glo (green), and DAPI (blue) bound to polymer spots. Fluorescent, phase contrast and SEM images of selected strong binding polymers (PA104 and PA531) are shown.

(b). Chart comparing binding of viable cysts in the hit arrays before (dark grey) and after (light grey) proteinase K treatment.

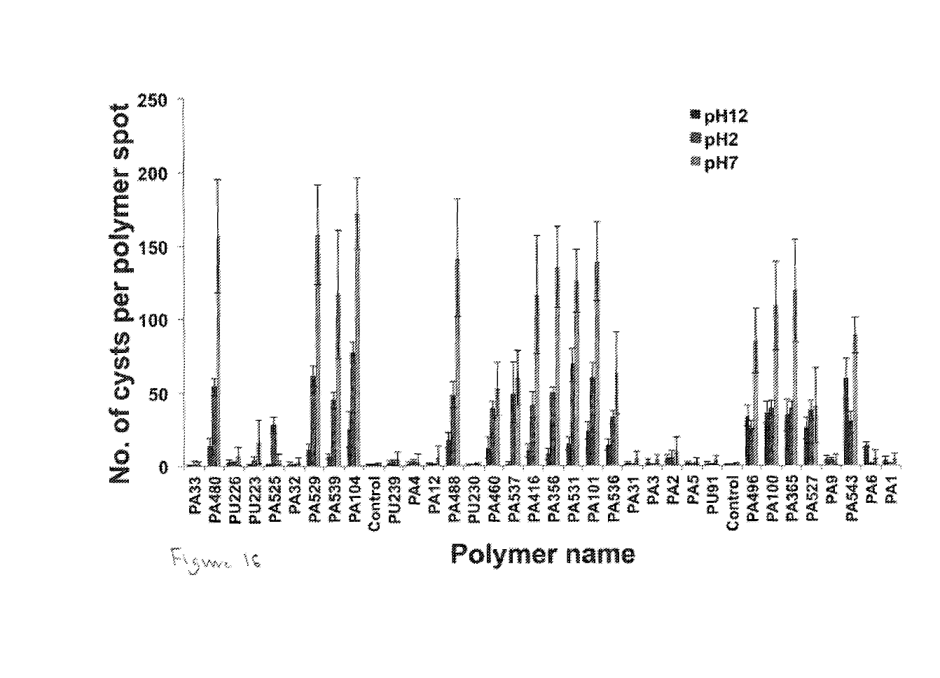

FIG. 16. Effects of pH on G. lamblia cyst binding. Chart of viable cyst binding at pH2 (light grey), pH7 (medium grey) and pH12 (dark grey).

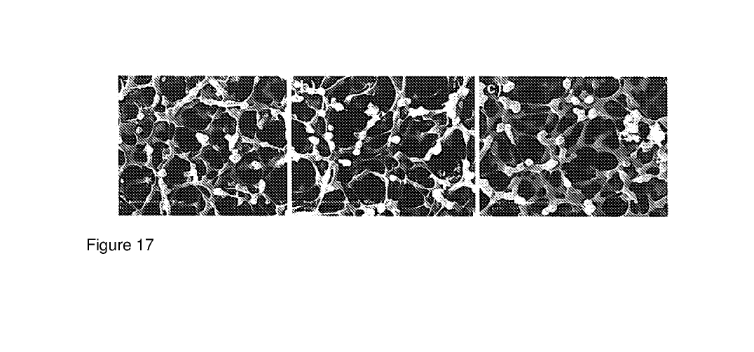

FIG. 17. Shows SEM images of (a) blank filter, (b) PA6 coated and (c) PA531 coated filters.

### MATERIALS AND METHODS

The polymers were synthesised as previously reported (3).

Scanning for C. parvum Oocyst Interactions

Cryptosporidium parvum (C. parvum) oocysts (Creative Science, Moredun, UK) or Giardia lamblia (G. lamblia) cysts (Waterborne Inc, USA) were diluted in sterilised water to a count of 1.66×10⁵ (oo)cysts per ml. When required, heat treatment of the samples for 5 mins at 70° C. was performed, using a Trechne Dri-Heat heating block, to obtain non-viable (oo)cysts. Loss of viability was confirmed using propidium iodide staining. Polymer microarrays were sterilised by exposure under UV light for 15 mins and freshly prepared 6 ml aliquots (1 million (oo)cysts per experiment) were added to a polymer microarray in a four-well plate. The slides were incubated with (oo)cysts on a plate shaker at 20-50 rpm for 3 hours at room temperature. Subsequently, the slides were rinsed with sterilised water and either fluorescently stained or prepared for SEM analysis.

Scale Up

Polymers were spin-coated onto circular glass coverslips (13 mm in diameter), incubated with C. parvum or G. lamblia (1.66×10⁵ (oo)cysts per ml in sterilised water) and imaged via brightfield and fluorescent microscopy as well as scanning electron microscopy (SEM).

Fluorescent Staining of C. parvum Oocysts and G. lamblia Cysts

The standard C. parvum and G. lamblia staining protocol (EPA1623) was adapted for the larger array area. After the slide was rinsed and air dried, 1 ml of methanol (MeOH) was added to the slide and allowed to air dry; 2 ml of 4′,6-diamidino-2-phenylindole (DAPI) (1 μg/ml) was applied to the slide for 1 min followed by a sterilised water rinse; finally, 2 ml of Crypto-a-glo was added to the slide (1-2 hrs) before rinsing in sterilised water and being left to air dry. A GeneFrame and a coverslip (1.9×6.0 cm, AB-0630) were then applied to each slide and cleaned with 70% ethanol. Image capture of the polymer microarray was performed via a Nikon 50i fluorescence microscope (20× objective) with an automated X-Y-Z stage, using the IMSTAR Pathfinder™ software package (IMSTAR S.A., Paris, France).

Results and Discussion

Results

The principle of high-throughput polymer array screening is illustrated in FIG. 1a. Briefly, pre-synthesised and characterised polymers were printed onto a glass slide, which was subsequently exposed to C. parvum oocysts or G. lamblia cysts. Following staining of the slides, automated screening was performed to capture images for each polymer with automatic counting of the number of (oo)cysts per polymer feature (initial array results as graphs shown in FIGS. 2-4 and hit array images shown in FIGS. 5-7).

For C. parvum and G. lamblia, polymer performance was maintained when scaled-up; with numerous C. parvum oocysts observed adhered to the PA531 coated surface in contrast to no oocysts on the surface of PA6 (FIG. 1d), while with G. lamblia (FIGS. 8 and 9) PA6 and PA32 prevented cyst adhesion and PA531, PA480 and PA104 promoting strong binding.

Scanning electron microscopy (SEM) was utilised to study the binding of both viable and non-viable oocysts on these selected polymers (FIGS. 10 and 11). SEM images of the large scale substrates coated with PA531 and PA6 were consistent with the polymer microarray results and fluorescent images of the coated surfaces (FIG. 1d and FIG. 11g, h, i, j). The morphologies of viable (FIG. 10c) oocysts on PA531 exhibited the expected oocyst features, with shape, size and presence of a central suture all in agreement with previous SEM studies of C.parvum oocysts (4). Occasionally differences in morphology were observed, with a higher proportion of non-viable oocysts having undergone excystation and release of their sporozoites (FIGS. 10d and 11c). SEM imaging (FIGS. 8 and 9) demonstrated the features expected of G. lamblia cysts, with their shape and sizes being consistent with results from previous studies (5). They also highlighted the differences between viable and non-viable cysts, with the walls being generally rougher and thicker in the latter.

Influence of Viability on C.parvum Adhesion

On the hit array, some polymers, such as PA531, PA528 and PA480, showed high binding for both viable and non-viable oocysts (FIGS. 5 and 6). Additionally polymers such as PA1, PA2, PA3, PA4, PA5 and PA6 completely prevented viable and non-viable oocyst adhesion (FIGS. 5 and 6). However, in general, notable differences in adhesion characteristics were observed in the results for viable and non-viable oocysts (FIG. 12). PA113 and PA531 were the top two polymers for adhesion of non-viable oocysts, while PA365 and PA464 demonstrated highest affinity binding for viable oocysts, perhaps indicative of different mechanisms, and relative strengths, of interactions. The polymers PA104 and PA504 demonstrated the highest selectivity in favouring of binding viable oocysts given that the ratio of viable oocysts to non-viable oocysts bound greater than 20 as opposed to an average of 4.5 for the selected hit polymer library (FIG. 12). A lower number of oocysts per polymer spot for the non-viable oocysts was observed, contradicting prior work which suggests that heat treatment of oocysts enables better adhesion via alteration/removal of surface glycoproteins (6). However, the influence of viability on oocysts adhesion has not previously been studied for polymer materials. Possibly, for polymer materials, the interaction is dominated by forces, such as hydrogen bonding or ion-pair interactions, and non-viable oocysts, with a reduced proportion of surface glycoproteins, are thus less able to interact with polymer surfaces. Comparison of the structures (FIG. 1c) of PA531 (strong interaction) and PA6 (inhibition of adhesion) supports this argument. PA531 comprises of MEMA (Methoxyethyl methacrylate) and DEAEMA (2-(Diethylamino) ethyl methacrylate) (FIGS. 1c, 13e) which contain several groups capable of participating in hydrogen bonding and ionic interactions, whereas PA6 is composed of styrene and DMAA (N,N-Dimethyl acrylate) (FIGS. 1c, 13e) and as such has a reduced capacity for these interactions.

Influence of Polymer Composition

For C. parvum, analysis of FIG. 13 shows clearly that specific chemical compositions inhibit binding and includes polymers containing styrene and DMAA (N,N-Dimethyl acrylate) or DEAA (N,N-Diethyl acrylate), while three out of four of those polymers which had the highest adherence of viable oocysts contained MEMA with DEAEMA or MEMA with DMAEMA (2-(Dimethylamino)ethyl methacrylate). We suggested that hydrogen bonding and acid-base interactions could play an important role in controlling surface adhesion of oocysts to polymers. The presence of MEMA and HEMA (2-Hydroxyethyl methacrylate), which have several sites to act as either hydrogen bond acceptors or donors, were found in many of the polymers selected for further analysis, supports this theory.

While knowledge relating to the exact composition of, and glycoprotein structures within, the oocyst wall is limited, the 5nm outer layer is believed to consist of acidic glycoproteins (6) and the ability of oocyst surfaces to form hydrogen bonds has been noted (7). Additionally, the presence of carboxylates and phosphates has been suggested by the fitted pKa value of 2.5 found by Karaman et al. (7). Our hypothesis is that hydrogen bonding, and acid-base interactions, play a key role in explaining the interaction of oocysts with polymer surfaces, and have more significant impact upon adhesion than hydrophobicity or surface roughness.

A key component of PA531 is DEAEMA, which has a reported pKₐ of 8.4 (8) which means that it will be protonated at all physiologically relevant pH's. This will thus ion-pair with the carboxylate/phosphate rich oocyst wall. The same argument holds for PA101 and PA480. The poor binding of PA1-6 can be rationalised by the non-charged nature of styrene and the acrylamides, DMAA and DEAA. Likewise, the PUs have no formal positive charge.

Several of the polymers in the G. lamblia hit array were identical or very similar to those selected for the C. parvum hit array, both for polymers which promoted, and those which prevented adhesion. This suggests that perhaps similar mechanisms control the adhesion of these two protozoan pathogens and some similarity between the composition of the oocyst and cyst outer walls. To investigate the relationship between chemical composition of the polymers and cyst adhesion, the monomeric composition was mapped against the results of the ‘hit’ array (FIG. 14), which indicated that inhibition of cyst binding was strongest in polyacrylates containing DMAA, DEAA or styrene, as well as selected polyurethanes. Monomers promoting strong binding were more variable; however the presence of DMAEMA, DEAEA 2-(Diethylamino)ethyl methacrylate (DEAEMA), or 2-(Dimethylamino)ethyl acrylate (DMAEA), was very common amongst the best performing polymers, such as PA104, PA480 and PA531.

Next, the nature of different functional groups present in the polymers was considered. For cellular adhesion it has been reported that glycol functionalities act in a preventative manner (9). This is normally attributed to the protein repellent nature of these moieties; for the majority of cell types adhesion is considered to occur via initial protein adsorption, which subsequently mediates cellular adhesion. For the protozoan experiments reported here prior protein interaction with the surface is not thought to be a possible mechanism of adhesion given that the experiments are performed in water and the cysts do not secrete proteins. However, the repellent nature of glycol functionalities is still consistent with our results, since none of the polyurethanes, containing monomers with glycols, exhibited strong interactions with cysts. In this case, the known poor likelihood of protein interaction with glycol moieties could apply to the cyst surface proteins, thus limiting any interactions between these polymers and the cyst outer wall.

A recent paper by Yang et al reported that aromatic functionalities were correlated with low cell adhesion whereas amine and ester moieties were found to promote cellular adhesion (9). The monomer most associated with low G. lamblia adhesion in the hit arrays was styrene, in agreement with the above finding that aromatic functionalities prevent adhesion. In terms of amine functionalities the monomers DMAEA, DEAEA, DMAEMA and DEAEMA, present in the ‘hit’ array in polymers also containing MEMA and MMA, all contain secondary amine groups and are associated with high levels of cyst adhesion. For cyst adhesion, the hypothesis is that at physiological pH values, the amines will be protonated and thus ion-pair with the cyst wall. DMAA and DEAA contain amide groups and are present in polymers which prevent adhesion. Since amide groups will not protonated at physiologically relevant pH this explains the lack of interaction with G. lamblia.

Influence of Proteinase K

To further understand the cysts surface interactions, viable cysts were treated with proteinase K, to remove proteins from the outer layers of the cyst wall, before analysis on a ‘hit’ array. The results showed that binding was severely limited for all polymers, with the number of cysts bound reduced by 70% compared to the untreated cysts (FIG. 15). Changes in morphology were also observed, with cysts appearing rounded with slightly thicker outer walls. Chatterjee et al (10) previously reported that removal of the cyst wall proteins decompresses the galactosamine fibrils, thus thickening the cyst wall. The reduction in adhesive ability suggests that the cyst wall proteins that bind the galactosamine fibrils play a crucial role in surface interactions. This supports the theory that protein specific interactions with polymers control the adhesion of cysts to these surfaces.

Influence of pH

Examining the ‘hit’ arrays at acid (pH 2) and base (pH 12) systems as opposed to the neutral system (pH 7), used in the standard arrays, provided very different results. While polymers demonstrating poor binding (less than 10 cysts per spot) in the previous ‘hit’ arrays showed little change, for those polymers previously shown to support adhesion the numbers of bound cysts was significantly reduced, with the average reduction, for the binding polymers, being 94% at pH 2 and 80% at pH 12 (FIG. 16).

In the previous discussion, the analysis of polymer composition and proteinase K treatment on cyst adhesion both suggested that ion-pair interactions play a key role in controlling the binding of G. lamblia to polymer surfaces. At pH2, below the isoelectric point for G. lamblia the cyst wall will be mainly positively charged and therefore will not react with the protonated amines. At pH12, while the cyst wall will be negative, the amines will be unprotonated and again no interactions will occur. Thus, performing experiments at different pH values significantly worsened the adhesive capacity of G. lamblia cysts to the polymers.

Coating Studies

Filters were coated using a solution deep coating method by dissolving 2% (w/v) polymer in three different solvents such as acetic acid (AA), tetrahydrofuran (THF) and acetone. The weight of each filter was recorded before immersion into the solution for 5 minutes. Wet filters were taken out from the solutions and dried under fume cupboard for 24 hours and the weight was recorded. The polymer loading was calculated using following relationship and the results are presented in Table 1. Results (Table 1) indicated that THF and acetic acid can be utilised for coating commercial filters (which was supplied by Idexx Filter-max filter). It was also found that the filters supplied by Idexx were partially dissolved in acetone, as indicated by weight loss (20-22%) (Table 2). Further characterisation of the coated filters was performed using SEM to investigate the uniform distribution of pore sizes. Considerable pore size variation was observed with no difference between the coated and uncoated filters (FIG. 17a-c). Therefore, any difference in performance of the coated filters would only be due to the surface chemistry rather than pore size changes.

% Polymer Loading=[(Final dry weight−Initial weight)/Initial weight]×100

TABLE 2
Results are showing the polymer loading onto filters using three
different solvents.

| | THF | | | ACETIC ACID | | | ACETONE | | |
|---|---|---|---|---|---|---|---|---|---|
| | I.W. g | F.W. g | P.L. % | I.W. g | F.W. g | P.L. % | I.W. g | F.W. g | P.L. % |
| PA-06 | 0.1699 | 0.1743 | 2.56 | 0.164 | 0.176 | 7.32 | 0.0779 | 0.0616 | −20.89 |
| PA-101 | 0.1724 | 0.1773 | 2.86 | 0.172 | 0.182 | 5.81 | 0.0931 | 0.0750 | −19.37 |
| PA-480 | 0.1722 | 0.1797 | 4.36 | 0.174 | 0.184 | 5.75 | 0.0771 | 0.0605 | −21.57 |
| PA-531 | 0.1713 | 0.1761 | 2.78 | 0.176 | 0.189 | 7.39 | 0.0954 | 0.0757 | −20.61 |

*Note:
I.W. = Initial weight,
F.W. = Final dry weight,
P.L. = polymer loading.

A filter housing system was set up to enable testing of the polymer coated filters and devised an appropriate testing protocol devised. This work confirmed the possibility of scale-up from polymer microarray system to filter coatings. The small-scale filter housing system using Millipore filter housing was set-up. Filters were cut to required size (˜13 mm) to fit the Swinnex filter holders and the uncoated side was marked. Filters were placed polymer-coated side up between the two O rings in filter holder. Solutions of Cryptosporidium were passed through the filter using a syringe. The waste water was also collected for further analysis, if needed. All filter samples were removed carefully from holders and washed gently in deionized water and dried, then stained with FITC-labelled antibody. From these initial trials it was difficult to distinguish the oocysts from the filter material due to high background levels of fluorescence from the polymer material taking up the stain. Thus, pre-labelled oocysts were subsequently utilised. Another challenge related to determining the concentration of oocysts in each sample. The experiments counted the number of oocysts bound to filters coated with different polymers, with the aim of comparing the capture efficiency of the different materials. Experiments showed that the polymers perform as expected, i.e. that high affinity coatings capture a high number of oocysts compared to the uncoated samples and that low affinity coatings result in better subsequent release of oocysts.

Recovery Studies

To achieve a high recovery rate of the Cryptosporidium oocysts, various elution protocols were trialled (Table 3 and Table 4). Initially, experiments were performed at different pH, using pre-labelled 100 oocysts counted by FACS. These samples were passed through the polymer coated and uncoated filters, and placed in 24 well plates. Solutions of 1 mL (with corresponding pH) were added to each well, and the whole plate was then incubated at 20 rpm on shaker for one hour. After incubation filter samples were rinsed gently, dried completely at room temperature, and fixed by adding 50 μL of methanol on each sample and dried. Oocysts immobilised filter samples were glued between two glass slides, ready for microscopic analysis. In order to recover oocysts the waste solution of different pH's was also kept, and filtered using Isopore Membrane Filters (Type 1.2 μm RTTP). The sample preparation technique for the microscopic analysis was as described above. The results are shown in Table 3, and indicate the alteration of pH is an effective method of increasing the elution from our polymer materials.

TABLE 3
pH dependent attachment behaviour of Cryptosporidium oocysts onto the
polymer coated filters and RTTP filter collected from the waste.

| | | | | Number of | | |
|---|---|---|---|---|---|---|
| | Number of | | | Cryptosporidium collected | | |
| | Cryptosporidium | | | from waste after pH | | |
| | remaining on the filter | | | treatment using 1.2 μm | | |
| | after pH treatment. | | | RTTP filter. | | |
| Filter Samples | pH 12 | pH 7 | pH 2 | pH 12 | pH 7 | pH 2 |
| Blank | 88 | 85 | 70 | 4 | 6 | 10 |
| PA 06 Coated | 65 | 75 | 35 | 8 | 10 | 20 |
| PA 101 Coated | 58 | 92 | 4 | 6 | 4 | 42 |
| PA 480 Coated | 4 | 95 | 5 | 22 | 2 | 15 |
| PA 531 Coated | 56 | 89 | 6 | 7 | 5 | 45 |

Results (Table 3) show the blank filter has no or small influence with the variation of pH studied. The polymer coated filters showed significant differences with the variation of pH, particularly coated with PA101 and PA531 (Table 3).

The elution study was also performed using a sonication approach. For this study the number of oocysts and the counting protocols were the same as the pH method, except the elution was performed at pH7 by placing the samples in a sonic bath. Each sample was placed in a single well in a 24 well plate and added 1 mL of pH7 buffer in each well, and then whole well plate was sonicated for two different time points. The protocols for the recovery of oocysts were same as the pH investigation and the results are presented in Table 4. Results (Table 4) show the sonication treatment, in the range studied, had no influence upon blank, PA480 and PA531 coated filters. A noticeable difference was seen in the case of PA101 and PA480 coated filters with the variation of treatment time in a sonic bath (Table 4).

TABLE 4
Cryptosporidium oocysts attachment behaviour of onto the
polymer coated filters and RTTP filter collected from the
waste as a function of the exposure time in sonic bath.

| | Number of | | Number of Cryptosporidium | |
|---|---|---|---|---|
| | Cryptosporidium remains | | collected using 1.2 μm | |
| | onto the filter after | | RTTP filter from waste | |
| | treatment in sonic bath. | | after treatment in sonic bath. | |
| Filter | 10 | 30 | 10 | 30 |
| Samples | minutes | minutes | minutes | minutes |
| Blank | 20 | 15 | 16 | 10 |
| PA 06 Coated | 85 | 5 | 12 | 19 |
| PA 101 Coated | 45 | 15 | 14 | 10 |
| PA 480 Coated | 52 | 56 | 11 | 22 |
| PA 531 Coated | 72 | 76 | 18 | 21 |

Synthesis of Selected Polymers in Large Scale (20-100 g).

The four polymers were selected for large scale synthesis as highlighted in the patent, which include PA06, PA101, PA480 and PA531.
1. Method of Synthesis of PA06: The synthesis of this polymer was performed using radical polymerisation method. The monomers, styrene (St) (mole ratio 50%) and N,N-dimethylacrylamide (DMAAm) (mole ratio 50%) were dissolved in toluene, and AIBN (2,2-Azo-bis-isobutyronitrile) was used as a radical initiator. The composition of monomers to solvent ratio was maintained at 20/80 (v/v).
2. Method of Synthesis of PA101: For the synthesis of this polymer, methoxyethyl methacrylate (MEMA) (50%) and 2-(dimethylamino)ethyl merthacrylate (DMAEMA) (50%) were dissolved in toluene, and AIBN was used as a radical initiator.
3. Method of Synthesis of PA480: For this polymer synthesis, methoxyethyl methacrylate (MEMA) (60%), 2-(diethylamino)ethyl merthacrylate (DEAEMA) (30%) and acrylic acid (AH) (10%) were dissolved in toluene, and AIBN was used as a radical initiator, and the monomers to solvent ratio was same as PA06.
4. Method of Synthesis of PA531: For the synthesis of this polymer, methoxyethyl methacrylate (MEMA) (55%) and 2-(diethylamino)ethyl merthacrylate (DEAEMA) (45%) were dissolved in toluene, and AIBN was used as a radical initiator.

For all polymers synthesis, the reaction temperature was maintained at 60° C. with prolonging for 24 hours, in a nitrogen gas purging environment. A precipitation and dissolution method was used to purify the polymers. Polymers were well characterised using GPC to ensure the molecular weights and distribution. Each polymer was synthesised with a scale of ˜50 gm.

TABLE 1
C. parvum hit array data analysis: number of viable/non-viable oocysts, polymer
wettability (water contact angle) and polymer surface roughness (root mean square).

| | No. of viable oocysts | No. of non-viable oocysts | Water contact | Root mean |
|---|---|---|---|---|
| Polymer | (per polymer spot) | (per polymer spot) | angle (°) | square (nm) |
| PA1 | 0 | 0 | 74 | 32.4 ± 4.98 |
| PA2 | 0 | 0 | 72 | 23.6 ± 3.28 |
| PA3 | 0 | 0 | 71 | 36.7 ± 3.43 |
| PA4 | 0 | 0 | 85 | 18.9 ± 3.11 |
| PA5 | 0 | 0 | 83 | 13.7 ± 3.28 |
| PA6 | 0 | 0 | 79 | 1.7 ± 0.024 |
| PA107 | 97.6 ± 7.33 | 42.6 ± 7.34 | 40 | 2.19 ± 0.067 |
| PA113 | 174.6 ± 24.43 | 96.1 ± 4.84 | 74 | 0.96 ± 0.027 |
| PA152 | 162.5 ± 9.19 | 56.4 ± 12.17 | 68 | 1.1 ± 0.051 |
| PA165 | 82.6 ± 22.71 | 37.3 ± 3.35 | 59 | 12.2 ± 0.75 |
| PA167 | 16.4 ± 1.50 | 11.5 ± 1.51 | 64 | 1.1 ± 0.040 |
| PA170 | 19.3 ± 0.860 | 14 ± 12.42 | 69 | 1.5 ± 0.022 |
| PA364 | 288.6 ± 15.96 | 21 ± 2.17 | 64 | 1.4 ± 0.037 |
| PA395 | 66.3 ± 5.89 | 20 ± 4.35 | 61 | 8.3 ± 0.45 |
| PA416 | 97.5 ± 6.08 | 26.4 ± 4.39 | 62 | 1.8 ± 0.11 |
| PA445 | 2.4 ± 0.102 | 0 | 66 | 2.4 ± 0.41 |
| PA464 | 253.5 ± 9.79 | 30 ± 7.69 | 69 | 6.7 ± 0.75 |
| PA476 | 59.1 ± 4.56 | 41.4 ± 7.97 | 74 | 7.2 ± 0.54 |
| PA480 | 223.9 ± 6.76 | 52.8 ± 10.0021 | 64 | 1.7 ± 0.12 |
| PA484 | 59.6 ± 4.05 | 22.7 ± 3.55 | 54 | 2.1 ± 0.12 |
| PA504 | 87.7 ± 3.21 | 3.9 ± 1.15 | 67 | 1.6 ± 0.090 |
| PA512 | 53.7 ± 2.08 | 10.9 ± 2.014 | 64 | 3.1 ± 0.19 |
| PA528 | 292.9 ± 4.67 | 42 ± 9.95 | 61 | 3.5 ± 0.18 |
| PA529 | 82.4 ± 4.09 | 19.2 ± 4.96 | 61 | 0.91 ± 0.0091 |
| PA531 | 255.9 ± 8.35 | 72.6 ± 6.88 | 61 | 2.0 ± 0.094 |
| PA539 | 208.1 ± 3.72 | 11.8 ± 2.90 | 60 | 5.6 ± 1.10 |
| PU91 | 0 | 0 | 82 | 17.0 ± 0.33 |
| PU223 | 2.3 ± 0.533 | 0 | 83 | 6.4 ± 1.10 |
| PU226 | 7.1 ± 0.427 | 0 | 83 | 0.73 ± 0.027 |
| PU230 | 0 | 0 | 32 | 59.0 ± 2.99 |
| PU239 | 1.4 ± 0.0521 | 0 | 28 | 8.80 ± 0.22 |

### REFERENCES

1. Smith H V, Nichols R A B. Cryptosporidium: detection in water and food. Experimental Parasitology. 2010; 124:61-79.
2. Dai X, Boll J, Hayes M E, Aston D E. Adhesion of Cryptosporidium parvum and Giardia lamblia to solid surfaces: the role of surface charge and hydrophobicity. Colloids and Surfaces B: Biointerfaces. 2004; 34:259-63.
3. Mizomoto H. The synthesis and screening of polymer libraries using a high throughput approach. Southampton: University of Southampton; 2004.
4. Poitras C, Fatisson J, Tufenkji N. Real-time microgravimetric quantification of Cryptosporidium parvum in the presence of potential interferents. Water Research. 2009; 43(10):2631-8.
5. Deng M Y, Oliver D O. Degradation of Giardia lamblia cysts in mixed human and swine wastes. Appl Environ Microbiol.58(8):2368-74.
6. Kuznar Z A, Elimelech M. Cryptosporidium Oocyst Surface Macromolecules Significantly Hinder Oocyst Attachment. Environ Sci Technol. 2006; 40:1837-42.
7. Karaman M E, Pashley R M, Bustamante H, Shanker S R. Microelectrophoresis of Cryptosporidium parvum oocysts in aqueous solutions of inorganic and surfactant cations. Colloid Surf A. 1999; 146:217-25.
8. van de Wetering P, Zuidam N J, van Steenbergen M J, van der Houwen O, Underberg W J M, Hennink W E. A mechanistic study of the hydrolytic stability of poly(2-(dimethylamino)ethyl methacrylate). Macromolecules. 1998 November; 31(23):8063-8.
9. Yang J, Mei Y, Hook A L, Taylor M, Urquhart A J, Bogatyrev S R, et al. Polymer surface functionalities that control human embryoid body cell adhesion revealed by high throughput surface characterization of combinatorial material microarrays. Biomaterials. 2010; 31(34):8827-38.
10. Chatterjee A, Carpentieri A, Ratner D M, Bullitt E, Costello C E, Robbins P W, et al. Giardia cyst wall protein 1 is a lectin that binds to curled fibrils of the GaINAc homopolymer. PLoS Pathogens. 2010; 6(8).

1-30. (canceled)

31. A method for identifying polymers which are capable of binding to protozoa, or are poorly binding and hence may be considered as non-binding to protozoa, comprising:
providing a library of polymer samples;
exposing the polymer samples to a target protozoa and
observing binding or non-binding of the target protozoa species to the polymer samples.

32. The method according to claim 31 wherein method is for identifying a polymer which is able to bind or repel Cryptosporidium and/or Giardia.species.

33. A method for the treatment or monitoring of water, so that protozoa may be removed or isolated from a sample of water, the method comprising contacting the sample of water with a polymer identified by the method according to claim 31.

34. An article comprising, consisting of, consisting essentially of, or coated with a protozoa binding polymer identified by the method according to claim 31.

35. A coating for a substrate, the coating comprising, consisting of or consisting essentially of a protozoan binding polymer or a protozoan non-binding polymer identified by the method according to claim 31.

36. The article according to claim 34 wherein the binding occurs under one defined condition (e.g. at a particular pH) and the protozoa are released by changing the condition from the condition used for binding (e.g. altering the pH).

37. An article, or coating comprising, consisting of, or essentially consisting of a polyacrylate/polyacrylamide polymer for use in binding protozoa.

38. The article, or coating according to claim 37 wherein a, or one of the monomers to make the polyacrylate/polyacrylamide polymer is a substituted alkyl methacrylate or acrylate monomer, wherein the alkyl group of the substituted alkyl function has from 1 to 10 carbon atoms and the substituent group is an alkoxy group with 1 to 4 carbon atoms.

39. The article, or coating according to claim 37 wherein the polymer comprises one or more of the following monomers, MEMA (2-methoxymethacrylate), DEAEMA (2-(diethylamino)ethyl methacrylate) and/or MEA (2-methoxyacrylate).

40. The article, or coating according to claim 37 wherein the polymer comprises a mixture of MEMA or MEA, and DEAEMA, in a ratio of 30:70 to 70:30.

41. The article, or coating according to claim 39 wherein the polymer further comprises one or more monomers such as acrylic acid (A-H).

42. The article, or coating according to claim 41 wherein polymer includes MEMA, DEAEMA and A-H in a ratio of 50:45:5 to 70:25:5.

43. The article, or coating according to claim 37 for use in binding Cryptosporidium and Giardia species wherein polymer comprises MEMA and DEAEMA in an amount of 50-60:50-30%, and further 10% of A-H.

44. An article, or coating comprising, consisting of, or consisting essentially of an acrylate or acrylamide/vinyl polymers, or polyurethane polymer for use in repelling protozoa.

45. The article, or coating according to claim 44 for use in repelling both Cryptosporidium and Giardia, wherein the polymer comprises one or more monomers which include an aryl group, a dialkylacrylamide group (alkyl representing 1 to 4 carbon atoms).

46. The article, or coating according to claim 44, wherein the polymer comprises two monomers selected from styrene and a dialkylacrylamide.

47. The article, or coating according to claim 46 wherein the polymer comprises, or consists of or essentially consists of the following monomers: St:DMAA and St:DEAA in the following ratios 45:55 to 95:5.

48. The article, or coating according to claim 37 for use in repelling Giardia, wherein the polymer comprises MEA (2-methoxyacrylate) and a dialkylacrylamide group (alkyl representing 1 to 4 carbon atoms).

49. The article, or coating according to claim 48 wherein the polymer comprises, consists of, or essentially consists of MEA:DMAA and MEA:DEAA, in the following ratios 45-75:55-25%.

50. The article, or coating according to claim 49 wherein the polymers are MEA(50%):DEAA(50%) and MEA(70%):DMAA(30%).

51. The article, or coating according to claim 37 for use in repelling protozoa, such as Cryptosporidium and Giardia species, wherein the polymer is a polyurethane which is formed by polymerising a polydiol with a di-isocyanate together with an extender molecule.

52. The method, article, or coating according to claim 51, wherein the extender is selected from the group consisting of:
PPG-PEG: poly(propylene glycol)-poly(ethylene glycol);
PTMG: poly(tetramethylene glycol) also known as poly(butylene glycol); and
PHNAD: poly[1,6-hexanediol/neopentyl glycol-alt-(adipic acid)]diol and
wherein the molecular weight of the extender is from Mn=600 to Mn=2500 and is present in an amount of 15-55%, of the polymer.

53. The article, or coating according to claim 51, wherein the di-isocyanate is selected from the group consisting of:
HDI: 1,6-diisocyanohexane;
MDI: 4,4′-methylenebis(phenylisocyanate); and
BICH: 1,3-bis(isocynanatomethyl)cyclohexane; and
the di-isocyanate is present in an amount of 45-55% of the polymer.

54. The article, or coating according to claim 51 wherein the extender is selected from the group consisting of:
BD: 1,4-butanediol;
OFHD: 2,2,3,3,4,4,5,5-octafluoro-1,6-hexanediol; and
DMAPD: 3-dimethylamino-1,2-propanediol;
present in an amount of 10-30 mol % of the polymer.

55. A method for the treatment of water or monitoring of water, so that protozoa may be removed or isolated from a sample of water, the method comprising contacting the sample of water with the article or coating according to claim 37.

56. A method for the treatment of water or monitoring of water, so that protozoa may be removed or isolated from a sample of water, the method comprising contacting the sample of water with the article or coating according to claim 44.
